(19) United States
(12) Patent Application Publication
(10) Pub. No.: US 2013/0017174 A1
(43) Pub. Date: Jan. 17, 2013

(54) COMPOSITIONS AND METHODS FOR INCREASING HEALTH AND REDUCING PATHOGENIC BACTERIA IN ANIMALS

(71) Applicants: Billy Hargis, Fayetteville, AR (US); Neil Pumford, Bentonville, AR (US); Amanda Wolfenden, Pea Ridge, AR (US)

(72) Inventors: Billy Hargis, Fayetteville, AR (US); Neil Pumford, Bentonville, AR (US); Amanda Wolfenden, Pea Ridge, AR (US)

(73) Assignee: The Board of Trustees of the University of Arkansas, Little Rock, AR (US)

(21) Appl. No.: 13/547,790

(22) Filed: Jul. 12, 2012

Related U.S. Application Data
(60) Provisional application No. 61/506,981, filed on Jul. 12, 2011.

(51) Int. Cl. A61K 35/74; A61K 33/00; A61P 31/04; A61P 1/00; A61P 37/06

(52) U.S. Cl. 424/93.3; 424/93.4; 424/718

(57) ABSTRACT

Probiotic compositions comprising a bacterium that is capable of producing nitric oxide and a substrate of nitric oxide synthase are provided. Also provided are methods of improving the gastrointestinal health of a subject by orally administering a bacterium capable of producing nitric oxide and orally administering a substrate of nitric oxide synthase. Methods of reducing the inflammatory immune response after treatment with a probiotic and methods of reducing horizontal transmission of pathogenic bacteria within groups of animals are also provided.

### CROSS-REFERENCE TO RELATED APPLICATIONS

This patent application claims the benefit of priority of U.S. Provisional Patent Application No. 61/506,981, filed Jul. 12, 2011, which is incorporated herein by reference in its entirety.

The present invention relates in general to improving the health of agricultural animals and reducing the chance of pathogenic bacterial contamination of food products by animal waste or during the slaughter process. In particular, probiotic formulations comprising bacteria and methods of using the same to improve the health of domestic animals, in particular poultry, are provided. The use of antibiotics in animal agriculture, in particular poultry production, is coming under increasing pressure from both consumers and government regulatory agencies.

This has created a need for effective antibiotic alternatives. The use of probiotics or direct-fed microbials (DFM) in animal agriculture may be one such potential alternative. The use of probiotics or DFMs may reduce the use of antibiotics in agricultural animal production and safeguard the food supply. In particular the use of probiotics or DFMs may improve the gastrointestinal health, increase weight gain, improve feed conversion ratios, and reduce the prevalence of pathogenic bacteria in the gastrointestinal tract of the animals.

### SUMMARY

Probiotic compositions or direct-fed microbials and methods of using probiotic compositions to increase the health of subjects, such as poultry, and reduce horizontal transmission to other animals and humans are provided herein. In one aspect, probiotic compositions including a bacterium capable Of producing nitric oxide and a substrate of nitric oxide synthase, such as nitrate or nitrite, are provided. The nitrate or nitrite may be provided such that the concentration in feed or water is between 10 ppm and 10,000 ppm.

In another aspect, methods of improving the gastrointestinal health of a subject are provided. The methods include oral administration of a bacterium capable of producing nitric oxide and of a substrate of nitric oxide synthase to the subject. The administration improves the gastrointestinal health of the subject as compared to a control.

In yet another aspect, methods of reducing the prevalence of pathogenic bacteria in the gastrointestinal tract of a subject are provided. The methods include oral administration of a bacterium capable of producing nitric oxide and a substrate of nitric oxide synthase such as nitrate or nitrite to the subject. The administration reduces the prevalence of pathogenic bacteria in the gastrointestinal tract of the subject.

In still another aspect, methods of reducing horizontal transmission of pathogenic bacteria in a group of subjects are provided. The methods include oral administration of a bacterium capable of producing nitric oxide and a nitric oxide synthase substrate such as nitrate or nitrite to the subject. The administration reduces horizontal transmission between subjects by reducing the pathogenic bacterial load in the group of subjects.

In a further aspect, methods of reducing the inflammatory immune response after treatment with a probiotic composition are provided. Suitably the probiotic composition comprises bacteria capable of producing nitric oxide. Alternatively bacteria capable of producing nitric oxide may be administered to the subjects in addition to the probiotic composition. In addition to the probiotic, a substrate of nitric oxide such as nitrate or nitrite is orally administered to the subject. Administration of the substrate of nitric oxide synthase is capable of reducing at least one immune effector, such as IL-2, IL-4 or IFN-γ production in the subject as compared to control subjects.

### BRIEF DESCRIPTION OF THE DRAWINGS

FIG. 1 is a graph showing the effects of adding a probiotic and various concentrations of nitrate on the growth of Salmonella enteritidis in vitro.

FIG. 2 is a graph showing the effects of adding a probiotic and various concentrations of nitrate on the growth of Salmonella enteritidis in vivo in either the crop or the cecal tonsils at 24 hours after treatment.

FIG. 3 is a graph showing the effects of adding a probiotic and various concentrations of nitrate on the growth of Salmonella enteritidis in vivo in tither the crop or the cecal tonsils at 72 hours after treatment.

FIG. 4 is a graph showing the effects of adding a probiotic and optionally 100 ppm of nitrate on the growth of Salmonella enteritidis in vivo in either the crop or the cecal tonsils at 24 hours after treatment.

FIG. 5 is a graph showing the effects of adding a probiotic and optionally 100 ppm of nitrate on the growth of Salmonella enteritidis in vivo in either the crop or the cecal tonsils at 72 hours after treatment.

FIG. 6 is a graph showing the effects of adding a probiotic and optionally 100 ppm of nitrate on the IL-4 mRNA levels in the ceca in vivo at 72 hours after treatment.

FIG. 7 is a graph showing the effects of adding a probiotic and optionally 100 ppm of nitrate on the IFN-γ mRNA levels in the ceca in vivo at 72 hours after treatment.

FIG. 8 is a graph showing the effects of adding a probiotic and optionally 100 ppm of nitrate on the IL-2 mRNA levels in the ceca in vivo at 72 hours after treatment.

### DETAILED DESCRIPTION

Recently, we determined that some of the key bacterial isolates in a commercially available probiotic, FloraMax-B11®, are capable of producing the powerful antimicrobial compound nitric oxide (NO). As nitrate substrates may be able to serve as precursors for NO synthesis by bacterial cells, we postulated that incorporation of nitrate or another nitric oxide synthase substrate into the medium, in the case of in vitro tests, or into chicken feed for in vivo testing, would increase the efficacy of this product on reducing pathogens such as Salmonella spp. The data provided in the Examples below describe the improved efficacy of this product in the presence of an added substrate of nitric oxide synthase both in vitro and in vivo.

Nitric oxide, a free radical gas, produced by phagocytes and other immune system cells has been shown to, have immunomodulating and antibacterial effects. As described in the Examples below, we found that a combination of the bacteria in the FloraMax-B11® probiotic and addition of nitrate, a substrate of the nitric oxide synthase, reduced the ability of Salmonella enteritidis to replicate both in vitro and in vivo. The results suggest that administering bacteria capable of producing nitric oxide in combination with a substrate of nitric oxide synthase, in particular a nitrate or nitrite, can increase the gastrointestinal health of animals and reduce the pathogenic bacterial growth or load in the subject's gastrointestinal tract. In addition, we expect that horizontal transmission of pathogenic bacteria from one animal to another within a group of animals would be reduced and that reduced pathogenic bacterial loads in animals will result in reduce contamination of animal-based food products including meat and eggs.

The methods may be carried out by orally administering a bacterium capable of producing nitric oxide and a nitric oxide synthase substrate, such as nitrate or nitrite to the subject. Oral administration can be by any known method including oral gavage, ingestion in feed or water or via any other means available to those of skill in the art. The bacteria capable of producing nitric oxide and the substrate of nitric oxide synthase can be administered in a single probiotic composition or may be administered separately. The bacteria may be administered before, at the same time or after administration of the substrate of nitric oxide synthase. The bacteria and the substrate of nitric oxide synthase may be administered in a single dosage form or may be administered continuously in the feed or water. In one embodiment, the bacteria are administered in a single dose and the substrate of nitric oxide synthase is provided continuously in the feed or water.

Bacteria capable of producing nitric oxide are known to those of skill in the art or can be determined using available tests for nitric oxide production. FloraMax-B11® contains several lactic acid bacteria capable of producing nitric oxide. Lactic acid bacteria or other bacteria capable of producing nitric oxide other than those in the FloraMax-B11® probiotic may be used in the compositions, and methods described herein. The bacteria may be provided in a probiotic composition or may be added to the feed or water provided to the subject.

Suitably, the substrates of nitric oxide synthase are nitrates or nitrites. The nitrates and nitrites may be provided in the form of a salt such as sodium nitrate used in the Examples. Other suitable salts include calcium nitrate, potassium nitrate, sodium nitrite or other salts of nitrate or nitrite. The nitrate or nitrite can be provided with the bacteria in a probiotic composition or alternatively may be provided separately in the feed or water. The nitrates and nitrites may be provided as a continuous supplement to the feed or water provided to the subject. In the Examples the nitrate was provided continuously in the feed at levels between 1 ppm and 1000 ppm. The nitrates or nitrites may be provided in feed or water at a concentration between 10 ppm and 10,000 ppm, suitably between 50 ppm and 1,000 ppm, suitably between 75 ppm and 500 ppm, suitably between 90 ppm and 200 ppm.

Probiotic compositions comprising a bacterium capable of producing nitric oxide and a substrate for nitric oxide synthase, such as nitrate or nitrite or a salt thereof are also provided.

The probiotic compositions may further comprise bacteria not capable of producing nitric oxide. The probiotic compositions may also include a pharmaceutically acceptable carrier. A pharmaceutically acceptable carrier is any carrier suitable for in vivo administration. Examples of pharmaceutically acceptable carriers suitable for use in the composition include, but are. not limited to, water, buffered solutions, glucose solutions, oil-based or bacterial culture fluids.

Additional components of the compositions may suitably include, for example, excipients such as stabilizers, preservatives, diluents, emulsifiers and lubricants. Examples of pharmaceutically acceptable carriers or diluents include stabilizers such as carbohydrates (e.g., sorbitol, mannitol, starch, sucrose, glucose, dextran), proteins such as albumin or casein, protein-containing agents such as bovine serum or .skimmed milk and buffers (e.g.,, phosphate buffer). Especially when such stabilizers are added to the compositions, the composition is suitable for freeze-drying or spray-drying. The composition may also be emulsified. Suitably the composition is formulated for inclusion in feed or water.

The bacteria capable of producing nitric oxide and the substrate of nitric oxide synthase may be administered in any order, at the same time or as part of a unitary composition. The two components may be administered such that one is administered before the other with a difference in administration time of a few minutes, 15 minutes, 30 minutes, 1 hour, 2 hours, 4 hours, 8 hours, 12 hours, 16 hours, 20 hours, 1 day, 2 days, 4 days, 7 days, 2 weeks, 4 weeks or more. The bacteria may be provided in dosage forms at regularly scheduled intervals or mixed in feed or water continuously. The substrate of nitric oxide synthase may be administered at regularly scheduled intervals or in feed and water continuously as well. In the Examples, the probiotic bacteria were administered in a dosage form by oral gavage and the substrate of nitric oxide synthase was administered continuously in the feed.

Administration of bacteria capable of producing nitric oxide with a substrate of nitric oxide synthase to subjects is capable of improving the health of the subjects after administration. In particular, the methods provided herein are capable of improving the gastrointestinal health of the subjects. This may include reducing the incidence or severity of necrotic enteritis (by at least 10%, 15% or even 20% as compared to controls), or reducing the bacterial load in the intestines of the animal, specifically with regards to levels of at least one pathogenic bacteria. As used herein, pathogenic bacteria include bacteria capable of causing disease in the subjects or in a human. Disease includes mortality, morbidity, or reduced productivity of agricultural animals, e.g., reduced weight gain, reduced offspring, egg or milk production, or reduced feed conversion ratio. For example the levels of Salmonella, Campylobacter, E. coli or Clostridium perfringens in the gastrointestinal tract of animals may be reduced (at least 50% decrease in recovery, suitably at least 60%, 70%, 80% or even 90% decrease in recovery as compared to controls). Improving the gastrointestinal health may also be quantified by an increase in the daily average weight gain of an animal (at least 3% increase, suitably at least a 5%, 7%, 10%, 20%, 30%, 40% or even 50% increase in weight gain as compared to controls over a set period of time such as a week or month). A suitable control is a similar subject not administered bacteria capable of producing nitric oxide with a substrate of nitric oxide synthase or the subject prior to administration of the bacteria and the substrate of nitric oxide synthase.

The methods may also reduce the level or number of potential bacterial food-borne pathogens of humans in the gastrointestinal tract of commercial agricultural animals as compared to controls (at least 50% decrease in recovery, suitably at least 60%, 70%, 80% or even 90% decrease in recovery as compared to controls). In particular, the level of Salmonella and Campylobacter spp. in the gastrointestinal tract of animals may be reduced in animals administered bacteria capable of producing nitric oxide and a substrate of nitric oxide synthase.

Such a reduction in potential human pathogen load in the gastrointestinal tract of animals will limit the opportunity of contaminating the human food chain either during preparation of meat for human consumption or via contamination of animal products such as poultry eggs. In addition, reduction of the pathogenic bacterial load in animals treated with, the methods described herein may also reduce horizontal transmission of pathogenic bacteria within a group of animals (suitably horizontal transmission is reduced by at least 10%, 20%, 30%, 40% or as much as 50%). As used herein, pathogenic bacteria include any bacteria capable of causing morbidity or mortality in the animal being treated using the methods described herein or in immunocompetent humans.

Suitably the subjects used in the methods are humans, mammals or poultry, suitably the animals are domesticated agricultural animals such as cows, pigs, sheep, or poultry, suitably a chicken or turkey. If supplied in an animal feed, the feed may comprise between 10⁵ and 10⁸ cfu total bacteria/gm of finished feed. Suitably the feed comprises between 10⁶ and 10⁷ cfu bacteria/gm feed. The probiotic formulation or the bacteria capable of producing nitric oxide and the substrate of nitric oxide synthase may be added to feed during production, after production by the supplier or by the person feeding the animals, just prior to providing the feed to the animals. The bacteria capable of producing nitric oxide and the substrate for nitric oxide synthase may be provided as a single dosage form, administered simultaneously, or administered sequentially or completely separately.

Methods of reducing the inflammatory immune response after treatment with a probiotic composition are also provided. The probiotic composition may include bacteria capable of producing nitric oxide or bacteria capable of producing nitric oxide may be administered in conjunction with the probiotic composition. Although probiotic compositions are generally used to increase the health of the animals being treated as well as reduce the number of pathogenic bacteria in the gastrointestinal tract of the animals, some probiotic treatments may induce an inflammatory immune response to the administered probiotic bacteria which may limit the benefit of the probiotic in terms of body weight gain. Reduced body weight gain may be associated with inflammatory immune responses in animals and may reduce the agricultural benefits of probiotic treatments. Addition of bacteria capable of producing nitric oxide and a substrate of nitric oxide synthase may reduce inflammation in the subject to which they are administered and may result in increased weight gain.

In the Examples, addition of nitrate to chicken feed at 100 ppm was shown to decrease the production of inflammatory mediators, specifically IL-2, IL-4 and IFN-γ. Thus, oral administration of a substrate of nitric oxide synthase to a subject, in combination with a NO-production-capable probiotic, may decrease the inflammatory immune response and increase the overall health of the subject in comparison to control subjects. Control subjects include subjects treated with a probiotic alone or untreated subjects. Immune effectors include but are not limited to cytokines or growth factors such as IL-1, IL-2, IL-4, IL-6, IL-10, TNF-α, IFN-γ, IFNα/β, TGF-β.

### EXAMPLES

In vitro Testing

Briefly, 1.25 grams of chick starter feed was measured into 13×100 mm borosilicate tubes and autoclaved. Nitrate was mixed into the feed before autoclaving at concentrations of 1000 ppm, 100 ppm, 10 ppm, or 1 ppm (Sodium Nitrate, Sigma Chemical Co., St. Louis, Mo.).

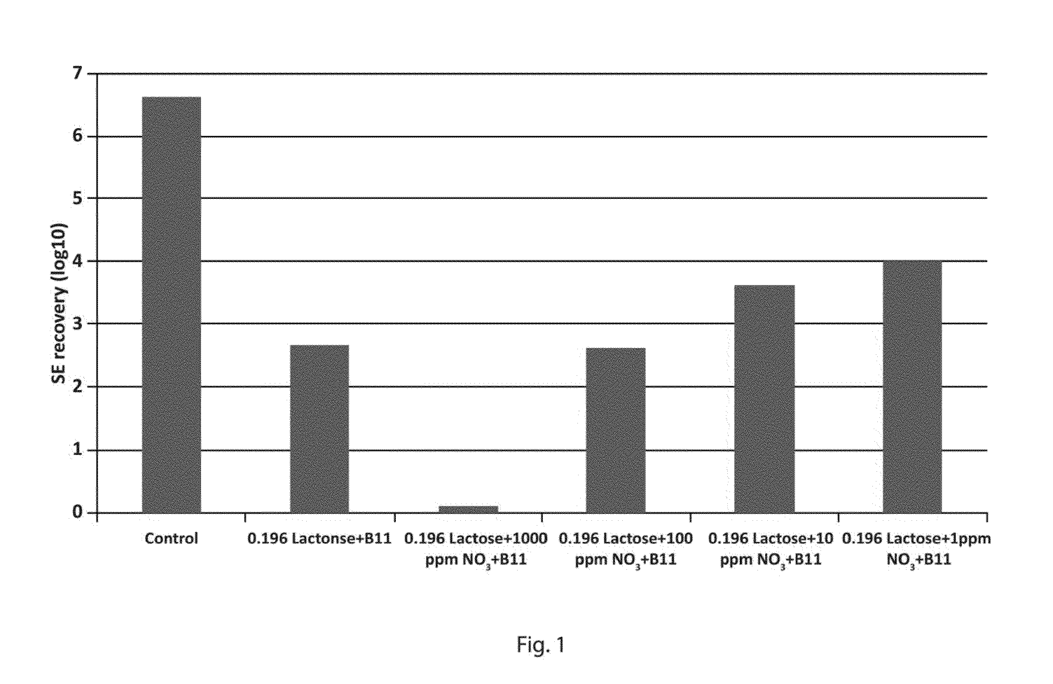

The feed also contained 0.1% lactose as a prebiotic. The feed was suspended in 4.5 mL sterile saline and inoculated with 0.5 mL of Salmonella enteriditis (SE) culture containing approximately 10⁴ cfu/ml. The tubes were treated with either 0.6 ml of 10⁶ cfu/ml of FloraMax-B11® probiotic or saline as a negative control. After administering the treatment, the tubes were agitated and incubated at 42° C. for 24 hours. The tubes then were agitated and the content was serially diluted and plated on Brilliant Green Agar (BGA) containing novobiocin (250 ml) and nalidixic acid (20 μl/ml) to select for SE. Typical SE colonies were counted after 24 hours of incubation. The results are shown in FIG. 1. Addition of increasing amounts of sodium nitrate resulted in decreased recovery of SE as compared to the control. In particular, addition of 100 ppm or 1000 ppm of nitrate with the FloraMax-B11® probiotic reduced the levels of Salmonella recovery equal to or beyond the reduction with the probiotic alone in the in vitro assay.

In vivo Testing

### Experiment 1

One-hundred and eighty day-of-hatch chicks were gavaged with 1.75×10⁴ cfu/chick of SE and randomly assigned to a group (n=30). One hour later chicks were gavaged with either 2.17×10⁷ cfu/chick of FloraMax-B11° probiotic or skim milk. Nitrate was included in the feed at either 1000 ppm, 100 ppm, 10 ppm, 1 ppm or 0 ppm according to treatment group throughout the testing period. Chicks were harvested at 24 and 72 hr after administration of the indicated treatment. Crop and cecal tonsils from 15 chicks/group were removed and enriched overnight in tetrathionate broth. Then samples were plated. on BGA containing novobiocin and naladixic acid as described above.

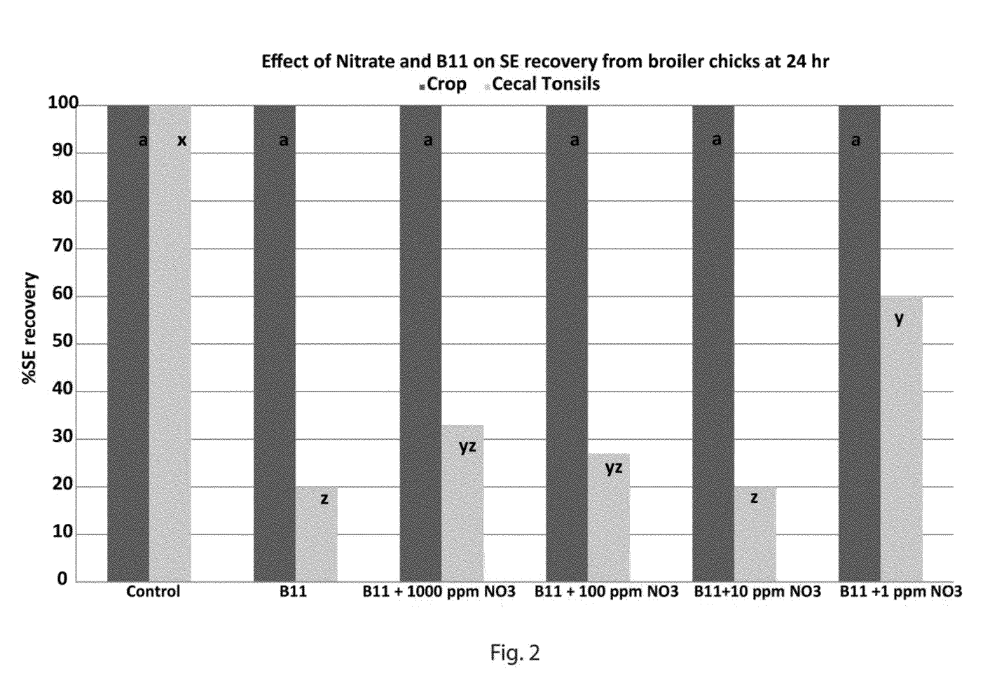

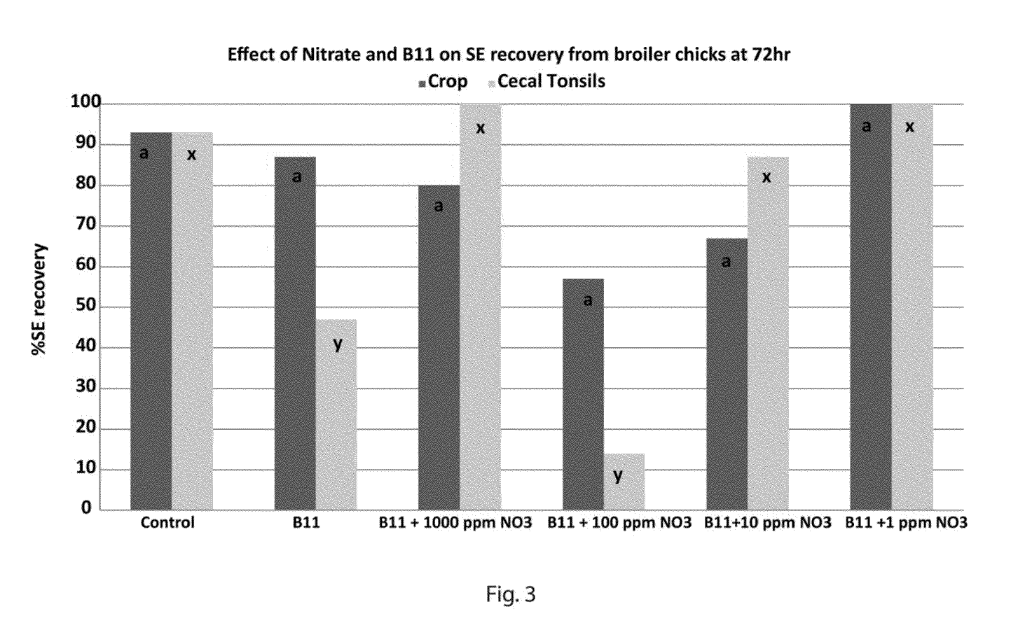

FIG. 2 and FIG. 3 show the resulting recovery of SE at 24 and 72 hours after administration. In the graphs different letters within sample types (i.e., crop or cecal tonsils) indicates significantly different values (P<0.05). As can be seen. in FIG. 2, administration of the probiotic alone or probiotic in combination with nitrate had no effect on the levels of SE recovered from the crop, but did significantly affect the levels of SE recovered from the cecal tonsils at 24 hours after administration. Notably the addition of nitrate had no effect at this time point beyond the probiotic alone. FIG. 3 shows that at 72 hours after administration, there was again no significant effect on SE recovery from the crop, but the trend in this preliminary experiment suggested that the crop levels of SE were slightly reduced after administration of the probiotic in combination with 100 ppm nitrate. Conversely, significant effects were observed in the levels of SE recovery in the cecal tonsils. Administration of the probiotic alone or in combination with 100 ppm nitrate reduced recovery of SE. While not statistically significant, the addition of notrate further reduced recovery of SE as compared to administration of the probiotic alone. These results suggest that administration of the probiotic reduces invasion of SE to deeper tissues from the gastrointestinal tract.

### Experiment 2

One-hundred and twenty day-of-hatch chicks were gavaged with 3.0×10⁴ cfu/ chick SE and randomly assigned to a group (n=40). One hour later chicks were gavaged with either 1.0×10⁷ cfu/chick of FloraMax-B11® probiotic or skim milk. Nitrate was included in the feed at either 100 ppm or 0 ppm according to treatment group. Chicks were harvested at 24 and 72 hr. Crop and cecal tonsils from 20 chicks/group were removed and enriched overnight in tetrathionte broth. Then samples were plated on BGA containing novobiocin and naladixic acid. Ceca from 10 chicks/group were collected for later mRNA isolation and analysis of IL-2, IL-4 and IFN-γ levels.

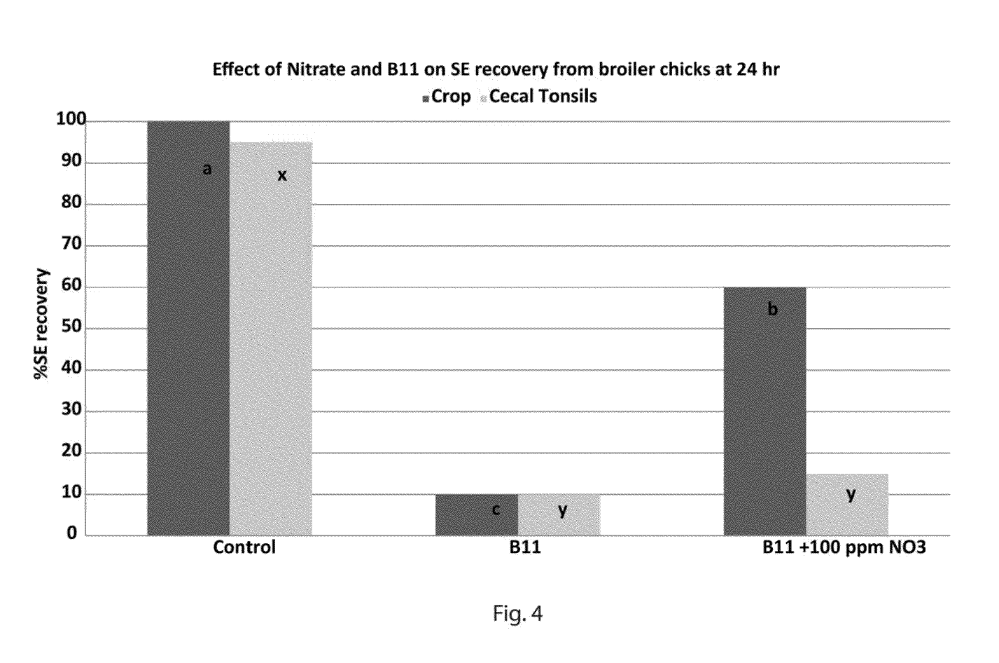

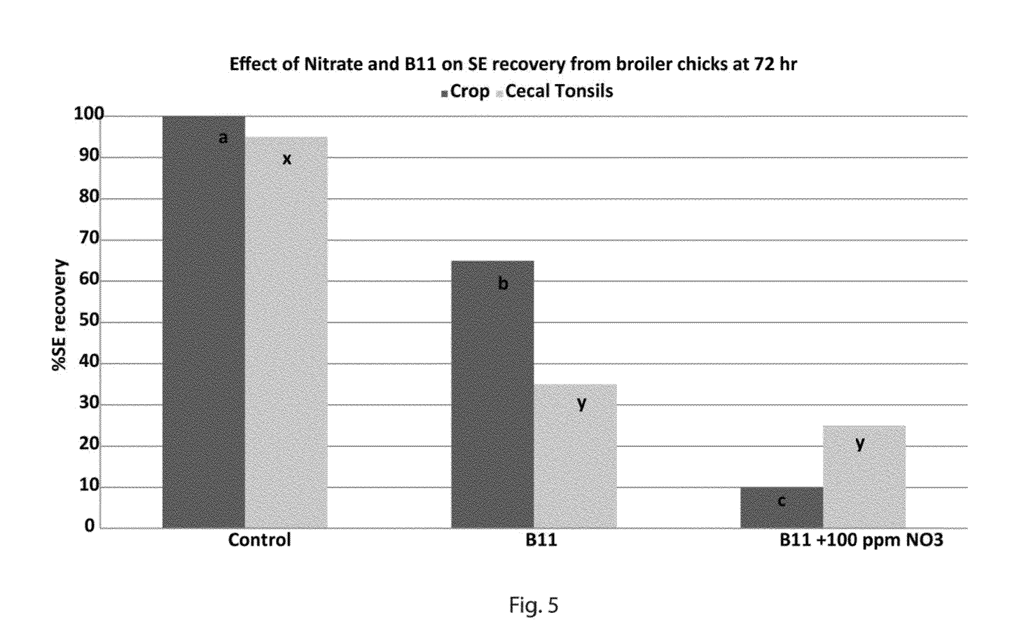

FIG. 4 and FIG. 5 show the resulting recovery of SE at 24 and 72 hours after administration. In the graphs different letters within sample types (i.e., crop or cecal tonsils) indicates significantly different values (P<0.05). As can be seen in FIG. 4, administration of the probiotic alone or probiotic in combination with nitrate had a statistically significant effect on the levels of SE recovered from the crop and the cecal tonsils at 24 hours after administration. No further improvement in reducing SE recovery was observed when the probiotic was administered with nitrate. In FIG. 5, administration of the probiotic alone or in combination with nitrate resulted in reduced SE recovery from both the crop and the cecal tonsils at 72 hours after administration. Notably, the addition of nitrate reduced SE recovery from both the crop and cecal tonsils at 72 hours post-administration. Thus, administration of the probiotic in combination with nitrate may result in a further increase in reduction of SE recovery particularly at later times after administration.

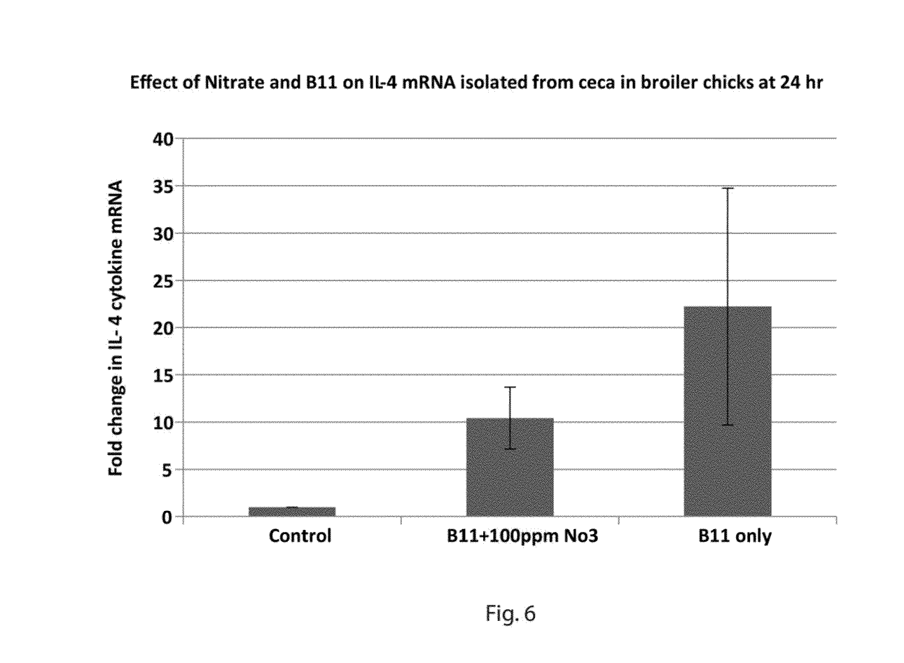

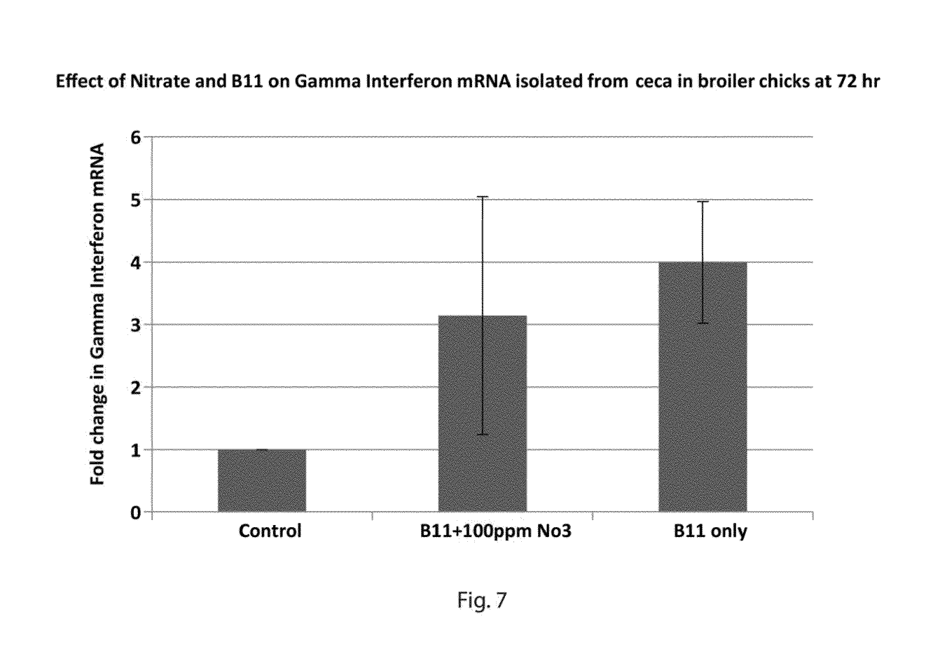

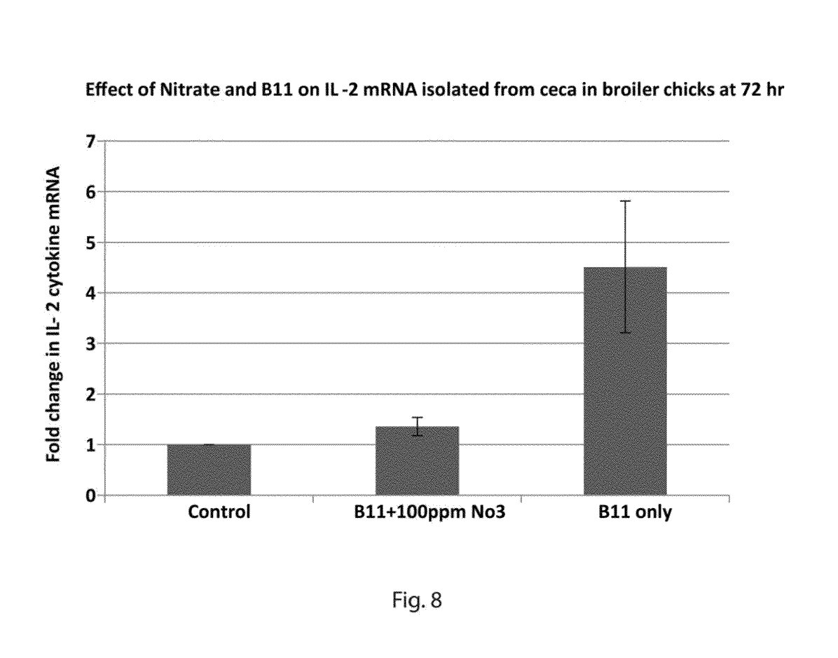

The results of the mRNA analysis of inflammatory mediators IL-4, IFN-γ and IL-2 are shown in FIGS. 6-8, respectively. FIGS. 6-8 shows that the levels of IL-4, IFN-γ and IL-2 are increased after administration of the probiotic as compared, to the control and that IL-4, IFN-γ and IL-2 levels are decreased after administration of the probiotic in combination with nitrate. These results suggest that the co-administration of the probiotic with nitrate results in a decreased inflammatory immune response to the bacteria in the probiotic. Inflammation is associated with decreased weight gain and reduced feed conversion ratios in agricultural animals.

1. A probiotic composition comprising a bacterium and a substrate of nitric oxide synthase, the bacterium capable of producing nitric oxide.

2. The probiotic composition of claim 1, wherein the substrate of nitric oxide synthase is a nitrate, nitrite or salt thereof.

3. The probiotic composition of claim 2, wherein the nitrite or nitrate concentration is between 10 and 10,000 ppm.

4. The probiotic composition of claim 1, wherein the bacteria are lactic acid bacteria.

5. The probiotic composition of claim 1, wherein the composition is formulated for administration in or with feed or water.

6. The probiotic composition of claim 1, wherein the composition further comprises bacteria not capable of producing nitric oxide.

7. A method of improving the gastrointestinal health of a subject comprising orally administering a bacterium capable of producing nitric oxide to the subject and orally administering a substrate of nitric oxide synthase to the subject, wherein administration improves the gastrointestinal health of the subject as compared to a control.

8. The method of claim 7, wherein the gastrointestinal health is improved by reducing the prevalence of pathogenic bacteria in the gastrointestinal tract of the subject, reducing the incidence or severity of necrotic enteritis, increasing the weight gain over time or improving the feed conversion ratio as compared to a control.

9. The method of claim 7, wherein the substrate of nitric oxide synthase is nitrate, nitrite or a slat thereof.

10. The method of claim 9, wherein the nitrate or nitrite concentration is between 10 and 10,000 ppm.

11. The method of claim 9, wherein the nitrate or nitrite concentration is between 50 and 1000 ppm.

12. The method of claim 7, wherein oral administration is in or with feed or water.

13. The method of claim 7, wherein the bacteria comprise lactic acid bacteria.

14. The method of claim 7, wherein the composition further comprises bacteria not capable of producing nitric oxide.

15. The method of claim 7, wherein the subjects are poultry.

16. The method of claims 7, wherein the bacteria and the substrate of nitric oxide synthase are administered at the same time.

17. A method of reducing the inflammatory immune response after treatment with a probiotic composition including bacteria capable of producing nitric oxide comprising orally administering a substrate of nitric oxide synthase to the subject, wherein administration of the substrate of nitric oxide synthase reduces at least one immune effector as compared to control animals.

18. The method of claim 17, wherein the immune effector is selected from IL-2, IL-4 and IFN-γ.

19. The method of claim 17, wherein the substrate of nitric oxide synthase is nitrate, nitrite or a salt thereof and the concentration is between 10 ppm and 10,000 ppm.

20. The method of claim 17, wherein the subjects are poultry.

21. A method of reducing horizontal transmission of pathogenic bacteria in a group of animals comprising orally administering a bacterium capable of producing nitric oxide to the animal and orally administering a substrate of nitric oxide synthase to the animal, wherein administration of the bacterium and the substrate reduces the pathogenic bacteria in the animal and thereby reduces horizontal transmission between animals in the group.

22. The method of claim 21, wherein the substrate of nitric oxide synthase is nitrate, nitrite or a salt thereof and the concentration is between 10 ppm and 10,000 ppm.

23. The method of claim 21, wherein the subjects are poultry.
